SEQUENCE LISTING (30 sequences)

SEQ ID NO: 1 - protein, 9 aa, synthetic construct
GGGGRGDSP

SEQ ID NO: 2 - protein, 10 aa, synthetic construct
CGGGGRGDSP

SEQ ID NO: 3 - DNA, 19 bp, synthetic construct
  source 1..19: mol_type = other DNA
acatcgctcagacaccatg

SEQ ID NO: 4 - DNA, 22 bp, synthetic construct
  source 1..22: mol_type = other DNA
tgtagttgaggtcaatgaaggg

SEQ ID NO: 5 - DNA, 21 bp, synthetic construct
  source 1..21: mol_type = other DNA
cttggccctgaggacattatt

SEQ ID NO: 6 - DNA, 22 bp, synthetic construct
  source 1..22: mol_type = other DNA
cacccaggtcagagttcaatag

SEQ ID NO: 7 - DNA, 22 bp, synthetic construct
  source 1..22: mol_type = other DNA
gggacttcagacccaagttatt

SEQ ID NO: 8 - DNA, 22 bp, synthetic construct
  source 1..22: mol_type = other DNA
cagacaggtgagctgattgtag

SEQ ID NO: 9 - DNA, 25 bp, synthetic construct
  source 1..25: mol_type = other DNA
agcagccgcatcttcttgtgcagtg

SEQ ID NO: 10 - DNA, 24 bp, synthetic construct
  source 1..24: mol_type = other DNA
ggccttgactgtgccgttgaattt

SEQ ID NO: 11 - DNA, 23 bp, synthetic construct
  source 1..23: mol_type = other DNA
gtcctagcctctttccaataaca

SEQ ID NO: 12 - DNA, 23 bp, synthetic construct
  source 1..23: mol_type = other DNA
gcatcttcccagatgagagtaaa

SEQ ID NO: 13 - DNA, 22 bp, synthetic construct
  source 1..22: mol_type = other DNA
gtctgtagctcattctgctctg

SEQ ID NO: 14 - DNA, 20 bp, synthetic construct
  source 1..20: mol_type = other DNA
gaaggcaactggatggaagt

SEQ ID NO: 15 - DNA, 19 bp, synthetic construct
  source 1..19: mol_type = other DNA
ctagaaaccccgaaccaaa

SEQ ID NO: 16 - DNA, 20 bp, synthetic construct
  source 1..20: mol_type = other DNA
ccagctatcaccaagctcgt

SEQ ID NO: 17 - DNA, 24 bp, synthetic construct
  source 1..24: mol_type = other DNA
gctctcactcgcccacttccttcc

SEQ ID NO: 18 - DNA, 24 bp, synthetic construct
  source 1..24: mol_type = other DNA
gccggactgctaatgccattggtt

SEQ ID NO: 19 - DNA, 17 bp, synthetic construct
  source 1..17: mol_type = other DNA
ctcgcttcggcagcaca

SEQ ID NO: 20 - DNA, 20 bp, synthetic construct
  source 1..20: mol_type = other DNA
aacgcttcacgaatttgcgt

SEQ ID NO: 21 - DNA, 50 bp, synthetic construct
  source 1..50: mol_type = other DNA
gtcgtatccagtgcagggtccgaggtattcgcactggatacgacaaccca

SEQ ID NO: 22 - DNA, 50 bp, synthetic construct
  source 1..50: mol_type = other DNA
gtcgtatccagtgcagggtccgaggtattcgcactggatacgacgacgta

SEQ ID NO: 23 - DNA, 50 bp, synthetic construct
  source 1..50: mol_type = other DNA
gtcgtatccagtgcagggtccgaggtattcgcactggatacgacgcggaa

SEQ ID NO: 24 - DNA, 23 bp, synthetic construct
  source 1..23: mol_type = other DNA
cggcggtgagaactgaattccat

SEQ ID NO: 25 - DNA, 18 bp, synthetic construct
  source 1..18: mol_type = other DNA
cgccgtctgggatgtgga

SEQ ID NO: 26 - DNA, 18 bp, synthetic construct
  source 1..18: mol_type = other DNA
cgcccgttcacagtggct

SEQ ID NO: 27 - DNA, 20 bp, synthetic construct
  source 1..20: mol_type = other DNA
ccagtgcagggtccgaggta

SEQ ID NO: 28 - protein, 5 aa, synthetic construct
PHSRN

SEQ ID NO: 29 - protein, 15 aa, synthetic construct
VSWFSRHRYSPFAVS

SEQ ID NO: 30 - protein, 19 aa, synthetic construct
ELTGAARKGSGRRLVKGPD

(19) United States
(12) Patent Application Publication
(10) Pub. No.: US 2023/0279379 A1
(43) Pub. Date: Sep. 7, 2023

(54) METHODS AND MICROFLUIDIC DEVICE FOR PRODUCING EXTRACELLULAR VESICLES

(71) Applicant: THE BOARD OF TRUSTEES OF THE UNIVERSITY OF ILLINOIS, URBANA, IL (US)

(72) Inventors: STEPHEN LENZINI, CHICAGO, IL (US); JAE-WON SHIN, CHICAGO, IL (US)

(73) Assignee: THE BOARD OF TRUSTEES OF THE UNIVERSITY OF ILLINOIS, URBANA, IL (US)

(21) Appl. No.: 18/178,736

(22) Filed: Mar. 6, 2023

Related U.S. Application Data
(60) Provisional application No. 63/316,586, filed on Mar. 4, 2022.

(51) Int. Cl. C12N 11/10; C12M 3/06; C12N 5/0775; C12M 1/00

(52) U.S. Cl. CPC C12N 11/10; C12M 23/16; C12N 5/0662; C12M 47/06; C12N 2533/74

(57) ABSTRACT

A method and microfluidic device for increasing the production of extracellular vesicles are provided which include the use of a cell-adhesive substrate for culturing extracellular-vesicle-producing cells, wherein the cell-adhesive substrate is a hydrogel having a Young's modulus of about 0.1 kPa to about 5 kPa and the hydrogel is functionalized with one or more cell adhesive peptides.

### INTRODUCTION

This invention was made with government support under grant nos. HL141255, HL125884 and HL007829 awarded by the National Institutes of Health. The government has certain rights in this invention.

This application claims the benefit of priority from U.S. Provisional Application Ser. No. 63/316,586, filed Mar. 5, 2022, the contents of which are incorporated herein by reference in their entireties.

### REFERENCE TO AN ELECTRONIC SEQUENCE LISTING

The contents of the electronic sequence listing (name: UIC0102US_ST26.xml; size: 27,408 bytes; and date of creation: Mar. 2, 2023) is herein incorporated by reference in its entirety.

### BACKGROUND

Extracellular vesicles (EVs) are cell-secreted nanomaterials composed of a lipid bilayer that are conventionally described as ranging from 50-500 nm in diameter. EVs have emerged as an important class of nanotherapeutics with broad potential to treat various tissue injuries, because they carry therapeutically relevant cargo molecules and deliver to recipient cells. EVs can also be readily modified to enhance targeting and therapeutic efficacy. Since EVs are naturally derived from cells, it is important to understand fundamental mechanisms of EV biogenesis in order to leverage them for manufacturing of therapeutic EVs. EVs are generally classified into subtypes based on their biogenesis pathways in cells. Microvesicles (or ectosomes) are produced by budding directly from the membrane, while intraluminal vesicles (ILVs) are produced by inward budding within late endosomes that form multivesicular bodies (MVBs). ILVs are then released as exosomes from cells when the MVB fuses with the plasma membrane. Recent studies have established EVs as a critical paracrine secretion mechanism for cell-cell communication. While physiologically relevant soluble signals, such as hypoxia, histamine, or Ca²⁺ levels are known to affect EV release, how cellular microenvironments regulate EV biogenesis remains largely unknown.

Mesenchymal stromal cells (MSCs) are known to utilize paracrine secretion mechanisms, including EVs to communicate with the host and control multiple inflammatory and degenerative conditions. Recent studies show that EVs from MSCs contain therapeutic cargo with bioactive contents that ameliorate lung injury, including angiogenic factors, growth factors, anti-inflammatory factors and mitochondrial contents such as mitochondrial DNA (mtDNA). MSCs respond to a variety of signals from microenvironments. In particular, advances in biomaterial design have revealed the sensitivity of MSCs to biophysical properties of the extracellular matrix, which in turn impact cell adhesion, spreading, and differentiation by modulating cytoskeletons. Previous studies suggest that actin cytoskeletons regulate biological processes that affect the plasma membrane surface area or mass, such as exocytosis and endocytosis (Diz-Muñoz et al. (2013) Trends Cell Biol. 23(2):47-53), which may inform the mechanisms of EV biogenesis. However, the role of matrix biophysical cues in mediating functional EV production at the nanoscale remains unclear.

Translation of EVs as treatments against tissue injuries will require efficient and scalable production of EVs ex vivo. Limitations on the number of EVs that can be produced for clinical studies have existed, especially if studies require autologous EVs. In addition, potential donor-to-donor variability of allogeneic EVs could negatively impact the reproducibility of therapeutic outcomes. Hence, there is a need for maximizing the yield of therapeutic EVs from a single donor source on a per cell basis. Maximizing the number of therapeutic EVs produced per cell by optimizing cell culture conditions is predicted to be the single most important driver of feasibility for producing EVs at large scales necessary for clinical translation. Thus, producing more EVs per cell in a given process will lead to a significant increase in clinical trial throughput, given that EVs maintain a similar therapeutic efficacy.

Previous efforts to improve EV productivity have included the use of small molecules (Wang et al. (2020) Cell 9(3):660), membranes or fibers (Colao et al. (2018) Trends Mol. Med. 24(3):242-256), hollow-fiber bioreactors (Watson et al. (2018) 7(1):1442088), and nanoporation (Yang et al. (2020) Nat. Biomed. Engineer. 4(1):69-83). However, alternative approaches are needed to provide increased EV production without compromising therapeutic efficacy in vivo.

### SUMMARY OF THE INVENTION

This invention provides a method of increasing the production of extracellular vesicles by adhering extracellular vesicle-producing cells (e.g., mesenchymal stromal cells) to a cell-adhesive substrate comprising a hydrogel having a Young's modulus of about 0.1 kPa to about 5 kPa, wherein the hydrogel is functionalized with one or more cell-adhesive peptides; incubating the extracellular vesicle-producing cells, optionally in the presence of one or more inhibitors of cell migration, actin polymerization, cell-cell adhesion, or cell-substrate adhesion; and collecting the extracellular vesicles secreted by the extracellular vesicle-producing cells. In some aspects, the extracellular vesicle-producing cells are adhered to the cell-adhesive substrate at a density of at least about 25 to about 150 cells per mm². In other aspects, the hydrogel is composed of alginate, chitosan, polyethylene glycol, polyacrylamide, gelatin, collagen, laminin, elastin, hyaluronic acid, decellularized scaffold from tissue, or a solubilized basement membrane preparation extracted from the Engelbreth-Holm-Swarm mouse sarcoma. In a further aspect, the one or more cell adhesive peptides include fibronectin peptides, hyaluronic acid peptides, decellularized scaffold from tissue, laminin peptides, peptides of a matricellular protein, linear RGD peptides, cyclic RGD peptides, PHSRN (SEQ ID NO:28) peptides, CD44-binding peptides, α6β1 integrin binding peptides, heparin-binding domain-derived peptides from thrombospondin, tenascin-C-derived peptides, or SPARC-derived peptides. In yet other aspects, the one or more inhibitors of cell migration, actin polymerization, cell-cell adhesion, or cell-substrate adhesion is an inhibitor of focal adhesion kinase, inhibitor of actin related protein 2/3, inhibitor of actin, inhibitor of a cadherin, or inhibitor of an integrin. In other aspects, step of collecting the extracellular vesicles is repeated 2 or more times in a 24 hour period or is carried out continuously.

This invention also provides a microfluidic device composed of one or more flow channels for collecting extracellular vesicles from extracellular vesicle-producing cells and a compartment including a cell-adhesive substrate composed of a hydrogel having a Young's modulus of about 0.1 kPa to about 5 kPa, wherein the hydrogel is functionalized with one or more cell adhesive peptides. In some aspects, the substrate has a thickness of about 5 μm to about 5 mm. In other aspects, the hydrogel is composed of alginate, chitosan, polyethylene glycol, polyacrylamide, gelatin, collagen, laminin, elastin, hyaluronic acid, decellularized scaffold from tissue, or a solubilized basement membrane preparation extracted from the Engelbreth-Holm-Swarm mouse sarcoma. In further aspects, the one or more cell adhesive peptides include fibronectin peptides, hyaluronic acid peptides, laminin peptides, peptides of a matricellular protein, linear RGD peptides, cyclic RGD peptides, PHSRN (SEQ ID NO:28) peptides, CD44-binding peptides, α6β1 integrin binding peptides, α3β1 integrin binding peptides, α6β4 integrin binding peptides, heparin-binding domain-derived peptides from thrombospondin, tenascin-C-derived peptides, or SPARC-derived peptides.

### BRIEF DESCRIPTION OF THE DRAWINGS

FIG. 1A-1B show that substrate elasticity determines the amount of EV secretion from MSCs. FIG. 1A, Primary human MSCs produce significantly more EVs on softer elastic alginate hydrogels conjugated with 0.8 mM RGD. Data represent the mean of N=3 experiments. *, p<0.05, ***, p<0.001 via one-way ANOVA with Tukey's post-test. FIG. 1B, Quantification of EV size distributions by nanoparticle tracking analysis. Line represents the median, box represents the 25^{th}-75^{th} percentile, and whiskers represent the 5^{th}-95^{th} percentile. Data represent the mean of N=3 experiments. For all, error bars indicate standard error of the mean (SEM).

FIG. 2 shows the results of treatment of primary human MSCs on soft and stiff substrates with 4 nM siRNA against talin and focal adhesion kinase (FAK) as compared to the scrambled control siRNA (SCR). *, p<0.05 via one-way ANOVA with Tukey's post-test, and data represent the mean of N=3 experiments. Error bars indicate SEM.

FIG. 3 shows that treatment of human MSCs with 3 μM MnCl₂ during and after adhesion to hydrogels inhibits EV secretion as compared to vehicle (veh) control. *, p<0.05, ***, p<0.001 via one-way ANOVA with Tukey's post-test, and data represent the mean of N=3 experiments. Error bars indicate SEM.

FIG. 4 shows that treatment of human MSCs with 200 nM EMD 121974 (EMD) during and after adhesion to hydrogels enhances EV secretion. *, p<0.05 via one-way ANOVA with Tukey's post-test, and data represent the mean of N=3 experiments. Error bars indicate SEM.

FIG. 5 shows that decreasing the amount of RGD conjugated in hydrogels significantly increases EV secretion. *, p<0.05, **, p<0.01 via one-way ANOVA with Tukey's post-test, and data represent the mean of N=3 experiments. Error bars indicate SEM.

FIG. 6 shows that blocking N-cadherin with an antibody (Ab) enhances EV production from a higher density cell culture on soft hydrogels as compared to the isotype control (control). *, p<0.05 via unpaired t-test. n=3 experiments. All error bars are SD.

FIG. 7 shows that softer hydrogel substrates facilitate intracellular MVB trafficking and fusion with the plasma membrane. Shown is ensemble <MSD> vs time plots. Data are fit to Equation 1. Error bars denote 95% confidence interval (CI).

FIG. 8 shows values of D_{0.4s} for tracks. *, p<0.0001 via unpaired Mann-Whitney test.

FIG. 9 shows that Arp2/3 limits EV secretion from MSCs on hydrogels by inhibiting MVB transport. Primary human MSCs treated with 5 μM CK869 (Arp2/3 inhibitor, CK) exhibit significantly increased EV secretion compared to DMSO (DM). N=3 experiments. *, p<0.05, ***, p<0.001 via one-way ANOVA with Tukey's post-test. Error bars denote SD.

FIG. 10 shows the effects of cell adhesion time on the kinetics of EV production from MSCs. The data were fit to the one-phase association equation, Y=Ym(1-e-kt), where Ym=EV/cell at the plateau and t1/2 (hr)=1n2/k. (Ym, t1/2) values for soft 4 hour=(22294, 0.23), soft 24 hour=(21142, 3.12), stiff 4 hour=(12309, 0.18), stiff 24 hour=(12458, 2.55). n=3 experiments. All error bars are SD.

FIG. 11 shows EV production kinetics upon depletion of EVs at 5 minute of culture after 4 hour adhesion of MSCs to soft and stiff hydrogels. (Ym, t1/2) values for soft=(22556, 14.4), stiff=(7076, 18.8). n=3 experiments. All error bars are SD.

FIG. 12 depicts a microfluidic device (10) for EV collection under continuous flow. Shown is a computer-aided design of a mold fabricated by 3D printing using a UV-curable, methyl methacrylate polymer.

### DETAILED DESCRIPTION OF THE INVENTION

Extracellular vesicles (EVs) are cell-secreted particles with broad potential to treat tissue injuries by delivering cargo to program target cells. However, improving the yield of functional EVs on a per cell basis remains challenging due to an incomplete understanding of how microenvironmental cues regulate EV secretion at the nanoscale. It has now been shown that cells capable of producing EVs (e.g., mesenchymal stromal cells (MSCs)) exhibit enhanced EV production when seeded on engineered hydrogels that mimic the elasticity of soft tissues. In particular, cells with a lower integrin ligand density seeded on a hydrogel having a Young's modulus of about 0.1 to about 5 kPa secrete ˜10-fold more EVs per cell than cells seeded on a rigid plastic substrate. Despite the difference in the number of EVs released, EVs remain similar in terms of their size, morphology, and expression of late endosomal markers. As cells adhere to substrates, integrins become increasingly activated, leading to a decrease in EV production. Notably, the EVs produced under such conditions can retain therapeutic cargo and therapeutic activity, as demonstrated in an acute lung injury model.

Therefore, the present invention provides a method for producing extracellular vesicles from extracellular vesicle-producing cells by adhering extracellular vesicle-producing cells to a cell-adhesive substrate comprising a hydrogel having a Young's modulus of about 0.1 kPa to about 5 kPa, wherein the hydrogel is functionalized with one or more cell adhesive peptides; incubating the extracellular vesicle-producing cells, optionally in the presence of one or more inhibitors of cell migration, actin polymerization, or cell adhesion; and isolating the extracellular vesicles secreted by the extracellular vesicle-producing cells.

As used herein, “extracellular vesicles” or “EVs” refer to membrane-bound structures released from or otherwise derived from cells. EVs include exosomes, microvesicles, apoptotic bodies, ectosomes, and high-density lipoprotein (HDL)-particles surrounded by a double lipid membrane structure and have various types of proteins (enzymes, growth factors, receptors, and cytokines), membranous lipids, nucleic acids and metabolites as their main contents. These structures are not limited in any way with regard to in vivo localization (e.g., intracellular or extracellular), in a body fluid, in a cell culture media, generated by in vitro cultured cells, mechanism of origin or size characteristics.

EVs can be derived, obtained, or isolated from a variety of producer cells. As used herein, the term “extracellular vesicle-producing cell” or “producer cell” refers to a cell used for generating an EV, e.g., an exosome. Suitable producer cells include stem cells such as embryonic stem cells (ESCs), induced-pluripotent stem cells, pluripotent stem cells, cord blood stem cells, amniotic fluid stem cells, progenitor cells, precursor cells and/or adult stem cells, e.g., neural stem cells, skin stem cells, epithelial stem cells, skeleton muscle satellite cells, mesenchymal stem cells, adipose-derived stem cells, endothelial stem cells, dental pulp stem cells (DPSCs), hematopoietic stem cells, stromal cells, endothelial precursor cells, or placenta-derived stem cells. More specifically, a producer cell includes a cell known to be effective in generating EVs, e.g., HEK293 cells, Chinese hamster ovary (CHO) cells, endothelial cells, mesenchymal stem or stromal cells (MSCs), BJ human foreskin fibroblast cells, fHDF fibroblast cells, AGE.HN® neuronal precursor cells, CAP® amniocyte cells, and RPTEC/TERT1 cells. In one aspect, EVs are derived from MSCs. In some aspects, EVs produced in accordance with the method of this invention include a biomarker. In accordance with this aspect, the biomarker may be a tetraspanin, such as, for example, CD63, CD9, CD81, CD82, CD53, and/or CD37. Other biomarkers include ADAM10, CD44, CD47, CD90 and/or AQP1.

Cells of use in the method of the invention may be passaged using conventional techniques and made into a single cell suspension within a medium such as Dulbecco's Modified Eagle Medium (DMEM), alpha-Minimum Essential Medium (MEM), or Iscove's Modified Dulbecco's Medium (IMDM). Medium may contain no additives, or additives such as standard or exosome-free fetal bovine serum (FBS) or human platelet lysate (hPL) as a supplement in an amount of between 0% to 10% w/v of the medium. Medium may also contain 1% penicillin/streptomycin antibiotics. Medium may also contain a glutamine supplement. Cells may be seeded on the cell-adhesive substrate or encapsulated in the cell-adhesive substrate at a density of at least about 25 to about 150 cells per mm². Cell density may be even higher if desired but is preferably not lower. Cells may be seeded for at least 4 hours (e.g., 6 hours, 8 hours, 10, hours, 12 hours, 14 hours, 16 hours, 18 hours, 20 hours, 22 hours, 24 hours or up to 48 or 60 hours) but preferably not longer than 3 days before exchanging medium to introduce a medium in cells will be cultured to produce EVs. Seeding for a sufficient amount of time allows cells to adhere to the cell-adhesive substrate. Washing the cell-adhesive substrate one or more times will remove non-adherent cells. Washing and exchange of medium may occur by aspirating existing medium and adding fresh medium. The wash medium may be, e.g., phosphate-buffered saline (PBS) or Hanks' Balanced Salt Solution (HBSS). The wash medium and fresh medium preferably do not contain any serum (FBS or hPL) supplements. The wash medium and fresh medium may be DMEM, alpha-MEM, or IMDM, and may contain 1% penicillin/streptomycin and/or a glutamine supplement. The cell-adhesive substrate may be cultured in the fresh medium for about 5 minutes up to 7 days before the conditioned medium containing EVs is collected and optionally isolated. In some aspects, EVs are collected 2 or more times (e.g., 3 times, 4 times, 5 times, 6 times, 7 times, 8 times or 10 times) in a 24 hour period from the medium in which the cells are being incubated. In other aspects, the EVs are continuously collected from the medium in which the cells are being incubated.

As used herein, the terms “isolate,” “isolated,” and “isolating” or “purify,” “purified,” and “purifying” and grammatical variants thereof are used interchangeably and refer to the state of a preparation (e.g., a plurality of known or unknown amount and/or concentration) of desired EVs, e.g., exosomes, that have undergone one or more processes of purification, e.g., a selection or an enrichment of the desired EVs. In some aspects, isolating or purifying as used herein is the process of removing, partially removing (e.g., a fraction) of the EVs from a sample containing producer cells. In some aspects, an isolated EV composition is enriched as compared to the starting material (e.g., producer cell preparations) from which the composition is obtained. This enrichment may be by at least about 10% to at least about 99% as compared to the starting material. Ideally, isolated EV preparations are substantially free of residual biological products (e.g., contaminants). In particular, the isolated EVs are about 90% to about 100% free of any contaminating biological matter. Residual biological products may include abiotic materials (including chemicals) or unwanted nucleic acids, proteins, lipids, or metabolites. In certain aspects, the isolated EVs are about 90% to about 100% free of any macromolecules, e.g., of any nucleic acids, proteins, lipids, and/or carbohydrates. Substantially free of residual biological products may also mean that the EV composition contains no detectable producer cells and that only EVs are detectable.

A “cell-adhesive substrate” refers to a substrate or surface that a cell may adhere to and/or otherwise be immobilized. In some aspects, cells that do not adhere may be removed by, e.g., washing. A suitable cell-adhesive substrate of use in the method of the invention includes a hydrogel having a Young's modulus in the range of about 0.1 kPa to about 5 kPa. A “hydrogel” refers to a substance formed when an organic polymer is crosslinked via covalent, ionic, and/or hydrogen bonds to create a three-dimensional open-lattice structure which entraps water molecules to form a gel. Examples of materials which can form hydrogels include natural matrix derivatives (e.g., from hyaluronic acid, decellularized scaffold from tissue, collagen, laminin, gelatin, elastin, and solubilized basement membrane matrix secreted by Engelbreth-Holm-Swarm (EHS) mouse sarcoma cells sold under the tradename MATRIGEL®), polyethylene glycol derivatives, polylactic acid, polyglycolic acid, polylactic-co-glycolic acid (PLGA) polymers, alginates and alginate derivatives, agarose, pectin, natural and synthetic polysaccharides, polyamino acids such as polypeptides particularly poly(lysine), polyesters such as polyhydroxybutyrate and poly-epsilon-caprolactone, polyanhydrides, polyphosphazines, poly(vinyl alcohols), poly(alkylene oxides) particularly poly(ethylene oxides), poly(allylamines) (PAM), poly(acrylates), modified styrene polymers such as poly(4-aminomethylstyrene), polyoxamers, poly(uronic acids), poly(vinylpyrrolidone), and copolymers of the above, including graft copolymers.

In some aspects, the hydrogels are crosslinked physically through divalent cations or covalently through click chemistry, thereby providing for a tunable complex modulus G*. Physical crosslinking leads to stress relaxing hydrogels and covalent crosslinking leads to elastic hydrogels. For purposes of this invention, a G* of about 500 Pa is deemed to be ‘soft’ and G* of about 3,000 Pa as ‘stiff’. Representative crosslinked hydrogels of the invention have a complex modulus G* in the range of about 0.1 kPa to about 5 kPa, or preferably about 0.3 kPa to about 4 kPa, or more preferably about 0.5 kPa to about 3 kPa, or most preferably 0.5 kPa±0.2 kPa. The crosslinked hydrogels of the invention may be nonporous or porous, e.g., nanoporous, like the decellularized matrix regardless of the crosslinking density or type. The crosslinked hydrogels possess a tunable range of stress relaxation behaviors. In certain aspects, the half-time of stress relaxation or stress relaxation rate is between about 1 second and 200 seconds, or more preferably about 10 seconds and about 100 seconds.

In some aspects, the hydrogel is alginate or modified alginate material. “Alginate” is a collective term used to refer to linear polysaccharides formed from (1-4)-linked 13-D-mannuronic acid monomers (M units) and L-guluronic acid monomers (G units) in any M/G ratio and sequential distribution along the polymer chain, as well as salts and derivatives thereof. The alginate monomers may be ionically and/or covalently crosslinked. In certain aspects, monomers are crosslinked with divalent or trivalent cation, e.g., Ca²⁺, Mg²⁺, Sr²⁺, Ba²⁺, Be²⁺, or Al³⁺. In other aspects, the divalent cation is Ca²⁺, Sr²⁺, or Ba²⁺.

In some aspects, the alginate monomers are covalently crosslinked. Representative covalent crosslinking methods include strain-promoted azide-alkyne cycloaddition (SPAAC), and click chemical reactions including the inverse electron demand Diels-Alder reaction between tetrazine and trans-cyclooctene (TCO), dibenzocyclooctene (DBCO) and azide, and tetrazine and norbornene. See Desai et al. (2015) Biomaterials 50:30-37 and references cited therein. One or a combination of ionic and covalent cross-linking can be used to modify the mechanical stability, stiffness, and/or utility of the hydrogel.

In some aspects, an alginate of use in the preparation of the hydrogel of this invention has a molecular weight of greater than about 40 kDa (e.g., about 41 kDa, about 120 kDa, about 250 kDa, about 300 kDa, about 400 kDa, or about 500 kDa). Preferably, an alginate of use in the preparation of the hydrogel of this invention has a molecular weight in the range of about 40 kDa to about 250 kDa.

In other aspects, hydrogels interpenetrated with other natural matrices such as collagen or the solubilized basement membrane matrix secreted by Engelbreth-Holm-Swarm (EHS) mouse sarcoma cells sold under the tradename MATRIGEL® may be prepared by mixing with the natural matrices in order to modulate (decrease) EV transport.

In accordance with the compositions and methods of this invention, the hydrogel is functionalized with one or more cell adhesive peptides. A hydrogel is deemed to be “functionalized” when the one or more cell-adhesive peptides are conjugated to the hydrogel. The cell-adhesive peptide is conjugated to the substrate in an amount sufficient to promote cell adhesion but insufficient to activate focal adhesion kinase. In some aspects, the cell adhesive peptide is conjugated to the hydrogel at a concentration of less than about 0.8 mM. In other aspects, the cell adhesive peptide is conjugated to the substrate at a concentration of about 1 μM to about 500 μM, or preferably about 10 μM to about 300 μM, or more preferably about 100 μM to about 200 μM. In some aspects, one or more cell adhesive peptides are derived from fibronectin, hyaluronic acid, laminin, or matricellular proteins. In other aspects, the cell adhesive peptide is a linear RGD peptide or a cyclic RGD peptide. In other aspects, the cell adhesive peptide is a Pro-His-Ser-Arg-Asn (PHSRN; SEQ ID NO:28) peptide. In other aspects, the cell adhesive peptide is a CD44-binding peptide. In other aspects, the cell adhesive peptide is a α6β1 integrin binding peptide (e.g., VSWFSRHRYSPFAVS; SEQ ID NO:29), α3β1 integrin binding peptides, or α6 4 integrin binding peptides. In other aspects, the cell adhesive peptide is a heparin-binding domain-derived peptide from thrombospondins (e.g., hep I; ELTGAARKGSGRRLVKGPD; SEQ ID NO:30). In other aspects, the cell adhesive peptide is a Tenascin-C-derived peptide. In other aspects, the cell adhesive peptide is a SPARC-derived peptide. In certain aspects, these peptides are covalently attached to the substrate by a coupling reaction.

As demonstrated herein, intracellular CD63⁺ multivesicular bodies (MVBs) transport faster within MSCs on softer hydrogels, leading to an increased frequency of MVB fusion with the plasma membrane to secrete more EVs. Actin-related protein 2/3 complex but not myosin-II limits MVB transport and EV secretion from MSCs on hydrogels. Thus, to further enhance EV production/secretion, the extracellular vesicle-producing cells may optionally be incubated in the presence of one or more inhibitors of cell migration, actin polymerization, cell-cell adhesion, and/or cell-substrate adhesion. In certain aspects, the extracellular vesicle-producing cells may be incubated in the presence an inhibitor of focal adhesion kinase (FAK), inhibitor of actin related protein 2/3 (Arp2/3), inhibitor of actin, inhibitor of a cadherin, and/or inhibitor of integrin. Exemplary blocking or neutralizing antibodies and small molecule inhibitors are presented in Table 1.

TABLE 1

| Target | Inhibitor | CAS No. |
|---|---|---|
| FAK | FAK-IN-1 | 2553215-22-6 |
| | FAK-IN-5 | 2408317-70-2 |
| | FAK-IN-7 | 19948-85-7 |
| | FAK-IN-8 | 1374959-91-7 |
| | FAK inhibitor 5 | 1426683-30-8 |
| | FAK inhibitor 2 | 2354405-14-2 |
| | Y15 | 4506-66-5 |
| | YH-306 | 1373764-75-0 |
| | Narmafotinib | 1393653-34-3 |
| | PF-573228 | 869288-64-2 |
| Arp2/3 | CK869 | 388592-44-7 |
| | CK666 | 442633-00-3 |
| | Benproperine phosphate | 19428-14-9 |
| | CK636 | 442632-72-6 |
| Actin | Cytochalasin D | 22144-77-0 |
| | Latrunculin A | 76343-93-6 |
| cadherin | NCD-2 | * |
| | ADH-1 or Exherin | 229971-81-7 |
| Integrin | JNJ-26076713 | 669076-03-3 |
| | SF0166 | 1621332-91-9 |
| | EMD 121974 | 188968-51-6 |
| | Intetumumab | 725735-28-4 |
| | MK-0429 | 227963-15-7 |
| | Etaracizumab | 892553-42-3 |
| | BG00011 (STX-100) | 1439902-67-6 |
| | SB-267268 | 205678-26-8 |

* Romeih et al. (2009) Dev. Growth Diff. 51(9): 753-67.

To facilitate production and isolation of EVs, the present invention also provides a microfluidic device that allows for the collection of EVs under continuous flow conditions. The microfluidic device of this invention is composed of one or more flow channels for collecting extracellular vesicles from extracellular vesicle-producing cells and a compartment comprising a cell-adhesive substrate, wherein the cell-adhesive substrate is a hydrogel having a Young's modulus of about 0.1 kPa to about 5 kPa, and wherein the hydrogel is functionalized with one or more cell adhesive peptides, as described herein. The cell-adhesive substrate is disposed inside a compartment of the microfluidic device to provide a soft surface on which to culture the EV-producing cells. In some aspects, the cell-adhesive substrate has a thickness of about 5 μm to about 5 mm.

A “microfluidic device” refers to a device through which materials, particularly fluids, such as wash medium or culture medium, can be transported on a micro-scale. In the exemplary illustration of FIG. 12, microfluidic device (10) is shown that includes a base layer or matrix (12) with a cell growth compartment (14) including a cell-adhesive substrate (16). In some aspects, the cell-adhesive substrate (16) is adhered to the base layer or matrix (12) of the microfluidic device (10). Fluid enters the microfluidic device (10) through an entry port (18) and exits the microfluidic device (10) through the exit port (20). Once introduced into the microfluidic device (10) via entry port (18), fluid is carried into and over the cell-adhesive substrate (16) of the cell growth compartment (14) through flow channels (22). As fluid passes over the extracellular vesicle-producing cells adhered to cell-adhesive substrate (16) of the cell growth compartment (14), EVs secreted by the extracellular vesicle-producing cells are collected by the fluid and exit the microfluidic device (10) via the exit port (20). Opening and closure of the flow channels (22) can be controlled, for example, through dot matrix style pin operated valves.

The microfluid device typically comprises structural or functional features dimensioned on the order of a millimeter-scale or less, which are capable of manipulating a fluid at a flow rate on the order of a μL/minute or less. The use of dimensions on this order allows the incorporation of a greater number of channels in a smaller area, and utilizes smaller volumes of fluids.

A microfluidic device can exist alone or can be a part of a microfluidic system which, for example and without limitation, can include: pumps for introducing fluids, e.g., samples, medium, buffers and the like, into the system and/or through the system; detection equipment or systems; data storage systems; and control systems for controlling fluid transport and/or direction within the device, monitoring and controlling environmental conditions to which fluids in the device are subjected, e.g., temperature, current, and the like.

The microfluidic device may be fabricated from glass or plastic such as polystyrene, polyvinylchloride (PVC), polytetrafluoroethylene (PTFE), and the like. The cell-adhesive substrate-contacting surface of the microfluidic device may be smooth or textured. The microfluidic device suitable for use in the present invention preferably has a hardness greater than the hardness of the cell-adhesive substrate disclosed herein, i.e., greater than about 5 kPa.

Other devices suitable for use in culturing EV-producing cells on the cell-adhesive substrate include cell culture vessels such as multiwell plates, bottles, flasks, petri dishes, and the like.

The EVs prepared in accordance with the method of this invention find particular use in EV treatment regimes. Accordingly, the invention also provides a method for using the EVs of this invention in EV therapy. In one aspect, an effective amount of a composition composed of EVs prepared from a soft gel matrix according to the method of the invention are administered to a subject in need of such treatment. As used herein, “subject” means an individual. Thus, subjects include, for example, domesticated animals, such as cats and dogs, livestock (e.g., cattle, horses, pigs, sheep, and goats), laboratory animals (e.g., mice, rabbits, rats, and guinea pigs) mammals, non-human mammals, primates, non-human primates, rodents, birds, reptiles, amphibians, fish, and any other animal. The subject is preferably a mammal such as a primate or a human. In some aspects, the subject has one or more damaged or dysfunctional cells and/or tissues. In a certain aspect, the subject is suffering from a lung injury. In particular aspects, the lung injury is endotoxin-induced acute lung injury, infection-mediated lung injury, or fibrotic lung injury.

According to some aspects, EVs can be used to treat a wide variety of cell types as well, including but not limited to immune cells, blood cells, vascular cells, epithelial cells, interstitial cells, musculature (skeletal, smooth, and/or cardiac), skeletal cells (such as bone, cartilage, and connective tissue), nervous cells (such as neurons, glial cells, astrocytes, Schwann cells), liver cells, kidney cells, gut cells, lung cells, skin cells or any other cell in the body.

Administration of a composition comprising EVs will be via any common route so long as the target tissue is available via that route. Such routes include oral, nasal, buccal, rectal, vaginal or topical route. Ideally, administration may be by intratracheal instillation, intratracheal inhalation, intravenous delivery, intramuscular delivery, intraarterial delivery, topical delivery, renal artery injection, portal vein injection, intrabone delivery, intraarticular delivery, intralymphatic delivery, intrathymic delivery, intrarenal delivery, intracorneal delivery, intraportal delivery, intrahepatic delivery, or intracardiac injection of the EVs. Such compositions would normally be administered as pharmaceutically acceptable compositions.

In some aspects, the EVs are delivered to and/or are taken up by damaged or dysfunctional cells and/or tissues. In several aspects, administration of the EV composition includes administration at a tissue or organ site that is the same as the target tissue. In some aspects, administration of EV composition involves administration at a tissue or organ site that is different from the target tissue. In some aspects, administration of EV composition includes administration systemically (e.g., in the blood).

In certain aspects, administration involves providing to a subject about 10², 10⁴, 10⁶, 10⁷, 10⁸, 10⁹, 10¹⁰, 10¹², or more EVs. In several aspects, a single dose of the EV composition includes between about 1×10⁶ and about 1×10⁹ EVs. In some aspects, a single dose of the EV composition is administered multiple times to the subject. The number of EVs administered may be chosen based on the route of administration and/or the severity of the condition for which the EVs are administered. In some aspects, the administration of the EV composition is inhalation or oral administration. In some aspects, the EV composition is administered by intra-arterial, intravenous, intratracheal, intrabone, or retrograde coronary sinus infusion or injection.

Tissues treated according the method provided herein include, in some aspects, cardiac tissue, brain or other neural tissue, skeletal muscle tissue, pulmonary tissue, arterial tissue, and capillary tissue. In several aspects, the tissue to be treated is damaged or dysfunctional is due to an injury, age-related degeneration, cancer, or infection. In some aspects, the methods provided herein treat tissue that is damaged or dysfunctional due to an acute event or a chronic disease. In some aspects, the acute event or chronic disease is as a result of myocardial infarction, traumatic head injury, and/or stroke. Non-limiting examples of additional chronic diseases that are treated include congestive heart failure, heart disease, ischemic heart disease, valvular heart disease, connective tissue diseases, HIV infection, dilated cardiomyopathy, myopathy, and dystrophinopathy (e.g., Duchenne muscular dystrophy), liver disease, sickle cell disease, dilated cardiomyopathy, infection such as Schistosomiasis, diabetes, Alzheimer's disease, Parkinson's disease, Huntington's disease, and Amyotrophic Lateral Sclerosis (ALS).

In additional aspects, the EVs are administered in conjunction with a therapeutic agent, e.g., an agent useful in treating the subject's disease or condition. The therapeutic agent is ideally within the lumen of the EVs and may include antibodies, proteins and peptides, nucleic acids, or small molecules.

Compositions containing the EVs can be prepared by combining the EVs with a pharmaceutically acceptable carrier or aqueous medium. The phrase “pharmaceutically acceptable” or “pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and the like. The use of such media and agents for pharmaceutically active substances is well known in the art. Except insofar as any conventional media or agent is incompatible with the EVs of the present disclosure, its use in therapeutic compositions is contemplated. Pharmaceutical compositions can be determined by one skilled in the art depending upon, for example, the intended route of administration, delivery format and desired dosage. See, for example, Remington, J. P. & Allen, L. V. (2013) Remington: The Science and Practice of Pharmacy. London, Pharmaceutical Press.

The EV compositions of the invention can be incorporated in an injectable formulation. The formulation may also include the necessary physiologically acceptable carrier material, excipient, lubricant, buffer, surfactant, antibacterial, bulking agent (such as mannitol), antioxidants (ascorbic acid or sodium bisulfite) and the like.

Acceptable formulation materials preferably are nontoxic to recipients at the dosages and concentrations employed. The pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the EV composition. Suitable formulation materials may include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA; complexing agents (such as caffeine, polyvinylpyrrolidone, beta-cyclodextrin or hydroxypropyl-beta-cyclodextrin); fillers; monosaccharides, disaccharides, and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as poloxamers, PEG, sorbitan esters, polysorbates such as polysorbate 20 and polysorbate 80, Triton™, trimethamine, lecithin, cholesterol, or tyloxapal); stability enhancing agents (such as sucrose or sorbitol); tonicity enhancing agents (such as alkali metal halides, preferably sodium or potassium chloride, mannitol, or sorbitol); delivery vehicles; diluents; excipients and/or pharmaceutical adjuvants. See, for example, Remington, J. P. & Allen, L. V. (2013) Remington: The Science and Practice of Pharmacy. London, Pharmaceutical Press.

The primary vehicle or carrier in a pharmaceutical composition may be either aqueous or nonaqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. Pharmaceutical compositions can comprise Tris buffer of about pH 7.0-8.5, or acetate buffer of about pH 4.0-5.5, which may further include sorbitol or a suitable substitute therefore. Pharmaceutical compositions of the invention may be prepared for storage by mixing the selected composition having the desired degree of purity with optional formulation agents (Remington's Pharmaceutical Sciences, Id.) in the form of a lyophilized cake or an aqueous solution.

The EV composition may be provided by sustained release systems, e.g., implantation devices. The EV compositions may be administered by bolus injection or continuously by infusion, or by implantation device. Where an implantation device is used, the device may be implanted into any suitable tissue or organ. The injections may be given as a one-time treatment, repeated (daily, weekly, monthly, annually etc.) in order to achieve the desired therapeutic effect.

The EV compositions of the invention may be delivered parenterally. When parenteral administration is contemplated, the EV compositions for use in this invention may be in the form of a pyrogen-free, parenterally acceptable aqueous solution. A particularly suitable vehicle for parenteral injection is sterile distilled water. Preparation can involve sustained release of the EVs, which may then be delivered via a depot injection. Formulation with hyaluronic acid has the effect of promoting sustained duration in the circulation. Implantable drug delivery devices may be used to introduce the desired composition.

These EV compositions may also contain adjuvants such as preservative, wetting agents, emulsifying agents and dispersing agents. Prevention of the action of microorganisms can be ensured by the inclusion of various antibacterial and antifungal agents, for example, paraben, chlorobutanol, phenol sorbic acid and the like. It may also be desirable to include isotonic agents such as sugars, sodium chloride and the like.

Supplementary active ingredients also can be incorporated into the EV compositions. The active compositions of the present disclosure may include classic pharmaceutical preparations. Administration of these compositions according to the present disclosure will be via any common route so long as the target tissue is available via that route. Such routes include oral, nasal, buccal, rectal, vaginal or topical route. Alternatively, administration may be by orthotopic, intradermal, subcutaneous, intraperitoneal, or intravenous injection. Such compositions would normally be administered as pharmaceutically acceptable compositions.

As used herein, the term “amount effective,” “effective amount” or a “therapeutically effective amount” refers to an amount of the EVs or EV composition of the invention sufficient to achieve the desired result. The amount of the EVs or EV composition which constitutes an “effective amount” or “therapeutically effective amount” may vary depending on the severity of the disease, the condition, weight, or age of the patient to be treated, the frequency of dosing, or the route of administration, but can be determined routinely by one of ordinary skill in the art. A clinician may titer the dosage or route of administration to obtain the optimal therapeutic effect.

Although not precluded, treating a disease or condition does not require that the disease, condition, or symptoms associated therewith be completely eliminated, including the treatment of acute or chronic signs, symptoms and/or malfunctions. “Treat,” “treating,” “treatment,” and the like may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition. “Treatment” therefore also includes relapse prophylaxis or phase prophylaxis. The term “treat” and synonyms contemplate administering a therapeutically effective amount of the EVs of the invention to an individual in need of such treatment. A treatment can be orientated symptomatically, for example, to suppress symptoms. Treatment can be carried out over a short period, be oriented over a medium term, or can be a long-term treatment, for example within the context of a maintenance therapy.

The foregoing may be better understood by reference to the following discussion and results which are presented for purposes of illustration and are not intended to limit the scope of the invention.

### Example 1: Materials and Methods

Material Preparation and Hydrogel Formation. Raw sodium alginates with different molecular weights, low (10/60, ˜120 kDa) and high (MANUGEL®, ˜240 kDa), were obtained from FMC Corporation. Alginate was purified through dialysis in a 3.5 kDa membrane submerged in water, followed by treatment with activated charcoal (Sigma) 0.5 g per gram alginate. It was then filtered, frozen and lyophilized to obtain a solid polymer. Conjugation of RGD (amino acid sequence GGGGRGDSP; SEQ ID NO:1, Peptide 2.0) to alginate polymers was performed using a method involving carbodiimide chemistry according to known methods at DS10 (0.8 μM) or DS2 (0.16 μM). Physically crosslinked hydrogels were formed by mixing 1% MANUGEL® and 1% 10/60 (2% total), adding to a syringe, and locking to another syringe with CaSO₄ (Sigma) to achieve final calcium concentrations of 10 mM (softer) and 25 mM (stiffer) (Lenzini et al. (2020) Nat. Nanotechnol. 15(3):217-223). After mixing, the solutions were deposited under glass for 2 hour to form a hydrogel. Covalently crosslinked hydrogels were formed using carbodiimide chemistry and adipic acid dihydrazide (AAD, Sigma). Alginate solution (1% MANUGEL® and 1% 10/60) was mixed with 4.8 mg/mL hydroxybenzotriazole (Sigma), 50 mg/mL 1-ethyl-3-(3-dimethylaminopropyl)carbodiimide (Sigma), and either 1.5 mM (soft) or 6 mM (stiff) AAD. Solutions were incubated under glass for 12-18 hours to form a hydrogel. polyethylene glycol-diacrylate (PEG-DA) hydrogels were formed by adding the materials: 10 mM sodium L-ascorbate (Sigma), 4 mM tris(2-carboxyethyl)phosphine (Sigma), 1X phosphate-buffered saline (PBS), 0.8 or 0.16 mM Cys-RGD peptide (sequence CGGGGRGDSP; SEQ ID NO:2, Peptide 2.0), PEG-DA Mn 700 (Sigma) and lithium phenyl(2,4,6-trimethylbenzoyl)phosphinate (TCI Chemicals) in varying concentrations to achieve desired range in mechanical properties upon 365 nm ultraviolet light exposure.

Mechanical Characterization of Hydrogels. The mechanical properties of hydrogels or tissues were obtained using rheometry via Netzsch Kinexus. Storage (G′) and loss (G″) moduli were measured through a frequency sweep by lowering the 8-mm plate geometry to a 5% normal strain followed by a rotation that induced a 0.5% shear strain at an increasing frequency and finally measurement of the resulting shear stress. The complex shear modulus G* was calculated:

G*=\sqrt{G′²+G″²}. (1)

Young's Modulus (E) was calculated with the equation:

E=2G*(1+v) (2)

using the value of G* obtained at 1 Hz, with Poisson's ratio v=0.5. To determine stress relaxation, the geometry was lowered at constant velocity (25 μm s⁻¹) through the linear elastic region until a 15% strain was reached, followed by measurement of normal force over time.

Cell Culture. Cells were cultured at 37° C. in 5% CO₂. Human MSCs (hMSCs) were derived by plastic adherence of mononucleated cells from human bone marrow aspirate (Lonza). After 3 days, adherent cells were cultured in the hMSC medium: α-minimal essential medium (aMEM, Thermo Fisher Scientific) supplemented with 20% fetal bovine serum (FBS, Atlanta Biologicals), 1% penicillin/streptomycin (P/S, Thermo Fisher Scientific), and 1% GlutaMAX™ (Thermo Fisher Scientific). After reaching 70˜80% confluence at 10˜14 days, cells were split, expanded in the hMSC medium and used at passage 3. D1 MSC cells (CRL-12424, ATCC) were cultured using high-glucose Dulbecco's Modified Eagle Medium (DMEM, Thermo Fisher Scientific) supplemented with 10% FBS, 1% penicillin/streptomycin (P/S, Thermo Fisher Scientific) and 1% GlutaMAX™ (Thermo Fisher Scientific) to 80% confluency before passaging, no more than 30 times. Cells were routinely tested for mycoplasma contamination and only used if no contamination was present.

Cell Seeding on Hydrogels. Hydrogel discs were placed in ultra-low binding polystyrene well plates (Corning) to ensure cells attach to hydrogels and not the plate surface. Hydrogels were washed with Hank's buffered salt solution (HBSS, Thermo Fisher Scientific) for at least 3 days before seeding cells. Cells were seeded at a low density (25 cells/mm²). After seeding, hydrogels were washed thoroughly to remove unattached cells. Drug treatments of 3 μM MnCl₂ (Thermo Fisher Scientific), 200 nM EMD 121974 sold under the tradename Cilengitide® (Cayman) and 5 μM CK869 (Cayman) were applied during and after cell adhesion to substrates. To evaluate the number of live cells seeded on substrates, cells were detached by incubating with a natural enzyme mixture with proteolytic and collagenolytic enzyme activity sold under the tradename ACCUTASE® Cell Detachment Solution (Innovative Cell Technologies, Inc.) for 10 minute at 37° C. Cells were then washed by centrifugation and directly added to HBSS containing calcein AM (1:2000; Biotium), ethidium bromide (1:2000; Thermo Fisher Scientific), and a predefined number of allophycocyanin (APC) beads (BD). After incubation at room temperature (RT) for 10 minutes, the samples were analyzed for live and dead cell number by flow cytometry.

Cell Morphology Analysis on Hydrogels. To evaluate cell morphology on substrates, cells were washed with HBSS, incubated with calcein AM (1:2000) for 10 minutes at 37° C., and imaged using a Nikon Eclipse Ts2R inverted fluorescence microscope. Cell circularity was calculated with the equation:

$$Circularity = \frac{4 π ( Area )}{{Perimeter}^{2}} . \qquad ( 3 )$$

Particle Size and Number Characterization. Particle size and number were obtained using Nanoparticle Tracking Analysis 3.2 (NTA) via NanoSight NS300 (Malvern) using a 405 nm laser. Samples were introduced by syringe pump at a rate 100 μL/min. Three thirty-second videos were acquired using camera level 14 followed by detection threshold 7. Camera focus, shutter, blur, minimum track length, minimum expected particle size and maximum jump length were set automatically by the software. Samples were diluted as needed to maintain particles per video from 100-2000. To ensure specificity, all samples were tested as compared to appropriate blank conditions.

Transmission Electron Microscopy. Samples were prepared by placing 10 μL onto a 300-mesh copper grid with carbon-coated formvar film (Electron Microscopy Sciences) and incubating for 2 minutes. Excess liquid was removed by blotting. Grids were placed briefly on 10 μL of 2% uranyl acetate, followed by blotting to remove excess liquid, and placed again. Grids were examined via JEOL JEM-1400F transmission electron microscope, operating at 80 kV. Digital micrographs were acquired using an AMT NanoSprint1200-S CMOS Camera and AMT software (Version 701). Particle diameter and sphericity were determined manually from images using ImageJ. Circularity of particles was defined by Equation 6.

Characterization of Late Endosomal Proteins in EVs. CD63 and CD9 expression were determined using in-house ELISA assays. Capture antibodies (CD63: BioLegend; CD9: BioLegend) were adhered to Nunc MAXISORP™ flat-bottom coated plates (Invitrogen) overnight followed by blocking for 1 hour with 1% bovine serum albumin (Roche) in PBS. After washing, samples were incubated overnight followed by incubation with biotin-conjugated detection antibodies (CD63: GeneTex; CD9: Miltenyi), incubation with Streptavidin-HRP (R&D Systems), and ELISA substrate (R&D Systems). Reactions were quenched with 1 M HCl and absorbance read at 450 nm. Recombinant protein standards (Sino Biological) were used as comparisons for protein content.

siRNA Transfection. Scrambled siRNA (Dharmacon) or siRNA against FAK (Ambion) or TLN1 (Dharmacon) was diluted to 160 nM in unsupplemented OPTI-MEM™ medium (Thermo Fisher Scientific) and combined 1:1 with OPTI-MEM™ supplemented with 2% transfection reagent sold under the tradename LIPOFECTAMINE® RNAiMAX (Thermo Fisher Scientific) and incubated at room temperature for at least 20 minutes. Cells were washed with HBSS and fresh growth medium was added to cells. The transfection solution was added dropwise for a final siRNA concentration of 4 nM to treat cells on hydrogels for 3 days followed by EV collection and measurement.

EV Isolation and Preparation. To isolate EVs from cells for cargo characterization and animal experiments, the cells were washed twice with HBSS followed by incubation with serum-free growth medium for 30 minutes. Afterward, the medium was exchanged with fresh serum-free medium. After times as indicated in the manuscript, medium was centrifuged at 2,000 g for 10 minutes to remove cell debris followed by centrifugation at 10,000 g to remove microvesicles (>500 nm) (Lobb et al. (2015) J. Extracell. Vesicles 4:27031). Afterwards, the supernatant was added slowly to a 14 ml, polystyrene ultracentrifuge tube (Beckman) containing 1.5 mL of 30% sucrose (Fisher Scientific) in PBS and centrifuged at 100,000 g for 90 minutes. The upper non-sucrose layer was aspirated and washed with PBS followed by centrifugation at 100,000 q for 90 minutes. The pellet was resuspended and confirmed to contain concentrated particles measured by NanoSight NS300 (Malvern).

EV Content Characterization. Before extraction, for all samples, particles were incubated with DNase I (Thermo Fisher Scientific) to remove potential exogenous DNA not contained within particles. DNA samples were treated with RNase A (Qiagen) to remove RNA contaminants, and DNA was extracted using the DNEASY® Blood and Tissue kit (Qiagen) followed by qPCR analysis. Total RNA was extracted from samples using the RNEASY® Mini kit (Qiagen). Complementary DNA was reverse transcribed from RNA by SUPERSCRIPT® III (Thermo Fisher Scientific). For both mRNA and miRNA, a random hexamer primer (Invitrogen) was used. For miRNA, an additional stem-loop RT (SLRT) primer was included at 100 nM for each specific miRNA target (TABLE 2).

TABLE 2

| | | SEQ ID |
|---|---|---|
| Target | Sequence | NO: |
| Human GAPDH | F: ACATCGCTCAGACACCATG | 3 |
| (NM_002046) | R: TGTAGTTGAGGTCAATGAAGGG | 4 |
| Human PTK2 (FAK) | F: CTTGGCCCTGAGGACATTATT | 5 |
| (NM_001199649.2) | R: CACCCAGGTCAGAGTTCAATAG | 6 |
| Human TLN1 | F: GGGACTTCAGACCCAAGTTATT | 7 |
| (NM_006289.4) | R: CAGACAGGTGAGCTGATTGTAG | 8 |
| Mouse GAPDH | F: AGCAGCCGCATCTTCTTGTGCAGTG | 9 |
| (NM_001289726.1) | R: GGCCTTGACTGTGCCGTTGAATTT | 10 |
| Mouse Fgf7 (KGF) | F: GTCCTAGCCTCTTTCCAATAACA | 11 |
| (NM_008008.4) | R: GCATCTTCCCAGATGAGAGTAAA | 12 |
| Mouse IL6 | F: GTCTGTAGCTCATTCTGCTCTG | 13 |
| (NM_001314054.1) | R: GAAGGCAACTGGATGGAAGT | 14 |
| Mouse ND1 | F: CTAGAAACCCCGAACCAAA | 15 |
| (NC_005089.1) | R: CCAGCTATCACCAAGCTCGT | 16 |
| Mouse mt-Atp6 | F: GCTCTCACTCGCCCACTTCCTTCC | 17 |
| (NC_005089.1) | R: GCCGGACTGCTAATGCCATTGGTT | 18 |
| Mouse Rnu6 (U6) | F: CTCGCTTCGGCAGCACA | 19 |
| (NR_003027.2) | R: AACGCTTCACGAATTTGCGT | 20 |
| SLRT for mouse | GTCGTATCCAGTGCAGGGTCCGAGGTATTCGC | 21 |
| miR-146A | ACTGGATACGACAACCCA | |
| (NR_029701.1) | | |
| SLRT for mouse | GTCGTATCCAGTGCAGGGTCCGAGGTATTCGC | 22 |
| miR-30b-3p | ACTGGATACGACGACGTA | |
| (NR_029534.1) | | |
| SLRT for mouse | GTCGTATCCAGTGCAGGGTCCGAGGTATTCGC | 23 |
| miR-27a-3p | ACTGGATACGACGCGGAA | |
| (NR_029746.1) | | |
| Mouse miR-146A | CGGCGGTGAGAACTGAATTCCAT | 24 |
| Forward | | |
| Mouse miR-30b-3p | CGCCGTCTGGGATGTGGA | 25 |
| Forward | | |
| Mouse miR-27a-3p | CGCCCGTTCACAGTGGCT | 26 |
| Forward | | |
| miRNA Universal | CCAGTGCAGGGTCCGAGGTA | 27 |
| reverse qPCR | | |
| primer | | |

F, Forward; R, Reverse.

Quantitative PCR was performed in the ViiA7 qPCR system with Power SYBR™ Green master mix (Applied Biosystems). Samples were analyzed using primer concentrations of 100 nM each with sequences in Table 2; for miRNA, the forward primer corresponds to the miRNA sequence and the reverse primer corresponds to the stem-loop sequence, which is universal for all targets. Relative gene expression was computed by the delta Ct method by comparing Ct values to a reference gene (GAPDH or U6). See Table 2 for the list of primers used. Samples were compared to a blank to ensure specificity of the assay. To analyze rRNA content, RNA samples were analyzed by Agilent TapeStation 4200 and 18S rRNA was considered as a peak in the range 1000-2000 nucleotides.

Lentiviral-Mediated Expression of CD63 Fused with Fluorescent Proteins. Katushka2S (far-red fluorescent protein) was fused with CD63 in a lentiviral expression vector (CD63-K2S vector) and expressed in D1 MSCs according to known methods. The sequence for pHLuorin2 (pH-sensitive green fluorescent protein) was synthesized by GenScript and exchanged with the Katushka2S in the same vector using restriction enzyme cloning. The resulting CD63-pHLuorin2 lentiviral vector was transduced in D1 MSCs in a similar method as the CD63-K2S vector. Cells were selected by treatment with 5 μg/mL puromycin over 3 days and confirmed to express fluorescent signal versus non-transduced cells.

Total Internal Reflection Fluorescence (TIRF) Imaging of Cells on Substrates. To covalently bond crosslinked PEG-DA hydrogel to a thin glass surface, acrylate groups were attached to glass coverslips by silanization. A solution of 3% v/v 3-(trimethoxysilyl)propyl acrylate (TCI Chemicals) and 5% v/v glacial acetic acid (Fisher Scientific) was prepared in methanol. No. 1 coverslips (Ted Pella) were incubated in the reaction solution for 45 minutes and thoroughly washed with methanol. The newly silanized coverslips were rinsed with ethanol and dried. A thin, flat (˜40 μm) layer of PEG-DA hydrogel was formed on the coverslips, followed by cell seeding, both as described above. Before imaging, cells were stained with an actin tracking stain sold under the tradename CELLMASK™ Green (Thermo Fisher Scientific) for Katushka2S experiments and a plasma membrane stain sold under the tradename CELLMASK™ Deep Red (Thermo Fisher Scientific) for pHLuorin2 experiments for 5 minutes at 37° C. followed by washing. Coverslip-hydrogels with seeded cells were mounted with immersion oil (Cargill) of refractive index 1.518 for Katushka2S experiments or 1.514 for pHLuorin2 experiments. Samples were imaged with a DeltaVision OMX SR microscope (GE) with an Olympus 60X Apo N objective. Dual channel 512×512-pixel (41×41 μm) images were obtained using the TIRF imaging mode with TIRF angle set at 80-90 degrees. For each cell, 250 images were obtained over 25 sec with frequency 100 ms per image.

CD63-Katushka2S Data Analysis. Using the IMARIS ‘Surfaces’ function, a custom tracking algorithm was created. Intracellular bodies were determined using Gaussian smoothing and local background intensity thresholding (with automatically determined thresholds) to detect surfaces followed by tracking their position (x, y) over time (t). To account for noise within images, bodies were discarded if they were constituted by less than 12 pixels (0.0768 μm²). Tracks could continue if the body was undetectable for a single timepoint within the track but not for two or more consecutive timepoints. Multivescular body (MVB) area was computed for each time t and reported as the mean area across total track time T. Tracks were then analyzed via a custom MATLAB (R2017b, MathWorks) program. Track MSD was calculated as

MSD(t)=[x(t)−x(t=0)]²+[y(t)−y(t=0)]². (4)

Ensemble-averaged track data were generated by averaging the MSD for each track i at every time t elapsed since the start of tracking:

$$< MSD ( t ) > = \frac{1}{N} {\sum }_{i = 1}^{N} {MSD}_{i} ( t ) \qquad ( 5 )$$

where N is number of tracks. For ensemble-averaged tracks, a lower limit of 15 points (1.5 sec) and an upper limit of 180 points (18 sec) were defined to constrain the tracks considered for analysis, as uneven track sizes can bias the results. Consequently, the ensemble-averaged data are shown only up to the lower limit (t=1.5 sec). Tracks were sorted into ‘Slow’ or ‘Fast’ populations using a threshold D_{0.4s}=0.001 μm²/sec.

CD63-pHLuorin2 Data Analysis. Flashing events were determined from image sequences using a custom MATLAB program. Potential event regions were determined by subtracting each image from a rolling average of the 5 previous images. The resulting image was converted to a binary image using a threshold of 40% of the mean image intensity. Regions identified after thresholding containing greater than 20 pixels (0.128 μm²) were considered. The total intensity within each region at the time of the event was then compared to the total intensity in the same region before the event (i.e., 5 images previous). A ratio of these intensities was taken, and the event was counted if the ratio exceeded three.

Sample Preparation for Imaging of F-Actin in Cells on Hydrogels. To prepare samples for F-actin imaging, glass bottom dishes (No 0., 35 mm; MatTek) were silanized, and a thin layer of PEG-DA hydrogel with varied elasticity was formed as described above. After cell seeding on hydrogels and culturing cells in the presence of DMSO or CK869, cells were permeabilized with 0.0015% saponin (Sigma) in a cytoskeleton buffer (10 mM MES, pH 6.0, 150 mM NaCl, 5 mM EGTA, 5 mM glucose, and 5 mM MgCl₂) for 4 minutes at room temperature. The samples were then washed with the cytoskeleton buffer 3 times and fixed with 4% paraformaldehyde in the cytoskeleton buffer for 20 minutes at room temperature. After washing 3 times with the cytoskeleton buffer, the samples were reduced in 0.1% sodium borohydride in PBS for 20 minutes at room temperature. To label F-actin in cells, the samples were stained with a fluorescent stain sold under the tradename ALEXA FLUOR® 488 phalloidin (1:1000; Cayman) for confocal imaging or ALEXA FLUORO 647 phalloidin (1:10000; Cayman) for super-resolution imaging in PBS with 3% BSA for 40 minutes at room temperature. The samples were washed 3 times with PBS prior to imaging.

Confocal Imaging of F-actin. Images of ALEXA FLUOR® 488 phalloidin-labelled cells on hydrogels were acquired by using the Zeiss LSM 770 confocal microscope with the 10X Plan-Apochromat objective by acquiring individual slices of the lateral plane at 1 μm increments along the axial plane with excitation/emission wavelength of 495/515 nm. A mean projection image was created from each confocal stack, and average intensity ALEXA FLUOR® 488 phalloidin per cell was analyzed by ImageJ (NIH). Each average intensity value was subtracted by the corresponding mean background intensity value from the same field of view.

Super-Resolution Imaging of F-actin. Super-resolution direct stochastic optical reconstruction microscopy (dSTORM) imaging was performed on a Nikon Ti inverted microscope with a 100X (NA 1.49) oil immersion objective in total-internal reflection (TIR) illumination condition with a 65 mW, 640 nm laser and an Andor IXON3 Ultra DU-897 EMCCD camera and collected in a far-red channel with EM filter 705/72. All dSTORM imaging ALEXA FLUOR® 647 phalloidin datasets were acquired using 80% excitation power setting at 10 ms exposure with the same EM gain after room temperature equilibration using NIS-Elements software. STORM buffer conditions used for oxygen scavenging were the same as described in the art. 50,000 images were recorded for each cell in each condition; after rejecting overtly dense/bleached images approx. 30,000 images were analyzed in each cell/condition for localization detection in post-processing using ThunderSTORM (ImageJ), with the following settings in brief: wavelet filtering (B-spline order 3, scale 2); integrated Gaussian fitting (radius: 3 pixels, initial sigma: 1.6 pixels); local maximum with 8-neighborhood; camera pixel size 160 nm. The localizations were then filtered via intensity to remove a majority of high uncertainty localization, followed by drift correction and removal of duplicate points.

Nanoscale Clustering Analysis of F-actin. The density-based spatial clustering of applications with noise (DBSCAN) algorithm was used to quantify clustering of F-actin localizations in each cell from dSTORM imaging (Rahbek-Clemmensen et al. (2017) Nat. Commun. 8(1):740) via a custom Python (3.8) program. The algorithm groups together localizations that are within a radius (ε), given a minimum number of localizations to be considered a cluster (ρ). Here, ε=half of average localization uncertainty for each cell (17.8±2.0 nm) to achieve a search radius that would enable at least two ALEXA FLUOR® 647 phalloidin localizations overlapping each other by the radius. The ρ value was set at 5 to remove any background signal outside the cell boundary. The F-actin clusters were classified and color-coded into 4 groups: (a) 5 or above, less than 15 (shorter fragments), (b) 15 or above, less than 100, (c) 100 or above, less than 1000, (d) 1000 or above (fiber-like structures). The percentage of F-actin localizations that belong to each cluster group per cell was then quantified and used for statistical analysis.

Measurement of Force for Membrane Tether Formation by Atomic Force Microscopy (AFM). Membrane tethering force was measured by using 3D-BIO AFM (Asylum Research, Oxford Instruments) with TR400PSA cantilevers (OTR4; spring constant=30 pN/nm). Indentation depth for each measurement was 4 μm with a velocity of 2 μm/s, and the force curve for each measurement was recorded by the AFM software (AR14). For each cell, 25-50 force curves were analyzed. In force mode, the piezotranducer (PZT) was set to drive the cantilever to approach, touch, make an indentation, and retract over a predefined distance in the optical axis perpendicular to the cell surface. The force curve was generated from the recorded vertical-axis movement of the PZT and the deflection of the cantilever with a known dimension and a spring constant. In the force curve representing the retraction of the cantilever from a cell, a sudden release of force occurred at the rupture of a membrane tether was quantified via a custom MATLAB program.

Animal Model of Acute Lung Injury. All animal procedures were performed in compliance with NIH and institutional guidelines approved by the ethical committee from the University of Illinois at Chicago. Female C57BL/6J mice were purchased from The Jackson Laboratory and housed in the University of Illinois at Chicago Biologic Resources Laboratory. At age 10-12 weeks, mice were treated with 10 mg/kg lipopolysaccharides (LPS, Sigma) via intraperitoneal injection to induce acute lung injury. After 4 hours, mice were anesthetized using ketamine/xylazine (50/5 mg/kg), and EVs (6×10⁷ per 20 g mouse) were administered by single dose intratracheal instillation. One day after LPS administration, mice were evaluated for lung vascular permeability and edema according to known methods. Briefly, mice were anesthetized using ketamine/xylazine (50/5 mg/kg) and a solution of Evans blue albumin (20 mg/kg) was applied via retro-orbital intravenous injection. After 20 minutes, mice were sacrificed and lung tissue was harvested along with a fraction of circulating blood. Right lung tissue was weighed initially (wet weight) and after 24 hours incubation at 65° C. (dry weight) to calculate wet/dry ratio. Left lung tissue was homogenized and Evans blue extracted with formamide. Evans blue content was measured by absorbance at 620 nm and normalized to that present in circulating blood. For CD44 blocking experiments, EVs (3×10⁸ per 20 g mouse) were incubated with 1 μg/mL of blocking CD44 antibody (BioXCell) or IgG control antibody (BioXCell) for 30 minutes at 4° C. prior to administration.

Statistical Evaluation. Statistics were performed as described in figure captions. All statistical analyses were performed using GraphPad Prism version 9.0.0. Unless otherwise noted, statistical comparisons were made from at least three independent experiments by one-way ANOVA followed by Tukey's multiple comparison test when standard deviations did not vary between test groups, and by one-way Welch ANOVA followed by Dunnett T3 multiple comparisons test when standard deviations were variable. For the datasets that do not show normal distributions based on the Anderson-Darling normality test, Mann-Whitney test was used to compare two groups, and Kruskal-Wallis test followed by Dunn's multiple comparisons test was used to compare more than two groups. A P-value less than 0.05 was considered statistically significant.

### Example 2: Substrate Elasticity Determines EV Secretion from MSCs

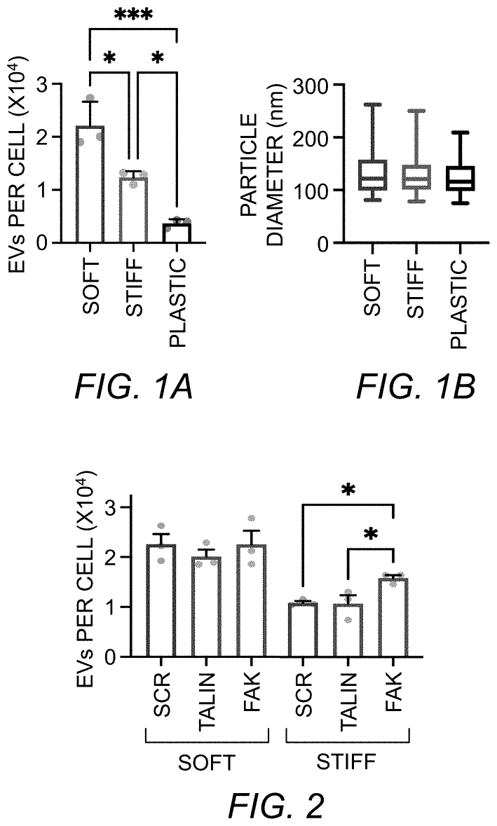

To test the effect of substrate mechanics on EV secretion from MSCs, hydrogel substrates were engineered which were composed of alginate polymer conjugated with the cell adhesion peptide Arg-Gly-Asp (RGD) that binds primarily to αᵥβ₃ and α₅β₁ integrins. Alginate-RGD hydrogels can be formed either covalently through adipic acid dihydrazide or physically through divalent cations, resulting in elastic or stress-relaxing hydrogels, respectively. For both types of hydrogels, elastic modulus (Young's modulus, E) of ˜3 kPa ‘soft’ and E ˜20 kPa ‘stiff’ were considered values that represent a physiologically relevant range of substrate elasticity for MSC mechanosensing, while E is ˜0.1 GPa for conventional plastic substrates. Primary human bone marrow MSCs were allowed to adhere to substrates for 24 hours followed by washout and collection of the serum-free conditioned medium from MSCs after 24 hours. After centrifugation of the medium at 10,000 g to remove cell, apoptotic, and larger particle fractions, particles remaining in the medium were analyzed by nanoparticle tracking analysis and normalized to the number of live adhered cells counted by flow cytometry (see Methods). MSCs were seeded at low density (˜25 cells/mm²) to avoid potential effects from cell-cell interactions. On elastic alginate hydrogels with 0.8 mM RGD, MSCs secreted significantly more particles on the soft hydrogel (˜20,000 particles/cell) than on the stiff hydrogel (˜10,000 particles/cell), while MSCs on a plastic substrate secreted 5-times less particles (˜4,000 particles/cell) than MSCs on the soft hydrogel (FIG. 1A). MSCs remain rounded on the soft hydrogel, while they show increased cell spreading and decreased circularity as the substrate becomes rigid, indicating that changes in EV secretion per cell as a function of substrate elasticity are associated with cell shape. Analysis of the particles pelleted after the 10,000 g centrifugation showed a lower number per cell and the number did not depend on substrate elasticity. Thus, focus was placed on the EVs remaining in the supernatant after the 10,000 g centrifugation. For these EVs, the particle size distribution remained similar across different substrates with median diameter ˜120 nm (FIG. 1B). Analysis of particle preparations by transmission electron microscopy showed that membrane-bound vesicles exhibit mean diameter ˜70 nm and circularity ˜0.75 regardless of substrates. Moreover, EVs from different substrates expressed similar levels of late endosomal markers CD63 and CD9. Importantly, levels of ribosomal RNA (rRNA) in particle preparations were similar across hydrogels or plastic substrates, and rRNA as a percentage of total RNA was lower than that found in untreated MSCs or particles derived from H₂O₂-treated apoptotic MSCs, ruling out apoptosis as a potential factor of increased EV secretion on the soft hydrogel. MSCs showed a similar level of EV secretion on faster stress-relaxing alginate-RGD hydrogels as elastic hydrogels, indicating that stress relaxation times of hydrogels do not impact EV secretion. The effect of substrate elasticity on EV secretion was also observed for primary mouse bone marrow MSCs and clonally-derived D1 mouse MSCs, as well as on RGD-bearing polyethylene glycol-diacrylate (PEG-DA) hydrogels with similar mechanical properties to elastic alginate hydrogels. Thus, substrate elasticity is an important determinant of EV secretion from MSCs without affecting vesicle size, morphology, or surface protein expression.

### Example 3: Inhibition of Outside-In Integrin-Ligand Interactions Promotes EV Secretion

The role of integrin-mediated cell adhesions on EV secretion for cells on hydrogel substrates was subsequently evaluated. The binding of talin to the cytoplasmic domain of integrin β₃ activates integrin inside-out signaling, while ligand binding induces outside-in signaling via focal adhesion kinase (FAK). To test roles of talin and FAK in EV secretion, human MSCs on elastic alginate-RGD hydrogels were treated with siRNA against talin or FAK over 3 days followed by evaluation of EV secretion. The knockdown efficiency of talin and FAK was ˜80%. While knockdown of talin expression had no effect, knockdown of FAK significantly increased EV secretion per cell on the stiff hydrogel (FIG. 2). These results indicate that integrin ligand-mediated focal adhesions limit EV secretion, which is enhanced on soft hydrogels (FIG. 1) where focal adhesions are less developed.

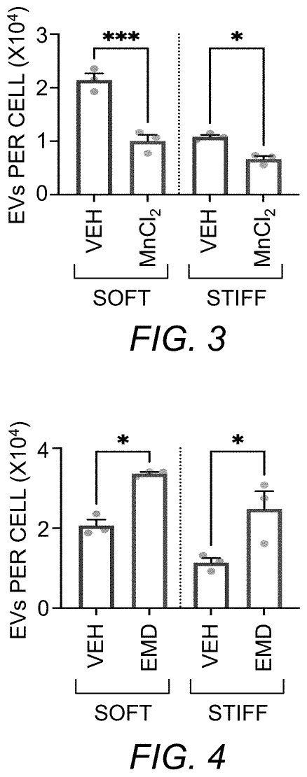

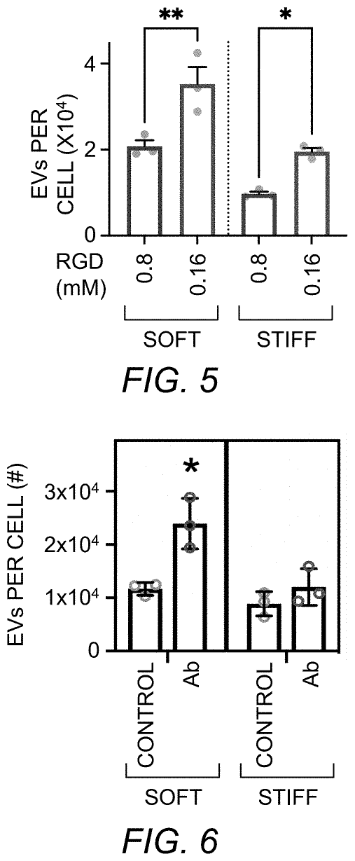

It was subsequently determined whether changes in integrin-ligand interactions on substrates were sufficient to influence EV secretion. When human MSCs were plated on elastic alginate-RGD hydrogels in the presence of 3 μM Mn²⁺, a treatment known to increase integrin-ligand affinity, EV secretion was decreased to a greater extent on the soft hydrogel than the stiff hydrogel (FIG. 3). In contrast, treatment of MSCs with EMD 121974, which interferes with integrin binding to RGD, significantly increased EV secretion on both soft and stiff hydrogels (FIG. 4). Neither treatment significantly affected the number of cells seeded on hydrogels or particle size. Consistent with these results, decreasing RGD concentration from 0.8 to 0.16 mM increased EV secretion per cell by ˜2-fold on both soft and stiff hydrogels (FIG. 5) while also maintaining the number of adhered cells per substrate and particle size. Thus, minimizing integrin-ligand interactions promotes EV secretion.

Similar to integrin-mediated cell adhesion, blocking of N-cadherin with an anti-N-cadherin antibody enhances EV production from a higher density cell culture on soft hydrogels (FIG. 6).

### Example 4: Softer Substrates Facilitate Intracellular MVB Trafficking and Fusion with the Plasma Membrane

To further understand how cell-matrix interactions mediate nanoscale events that result in EV secretion from MSCs, the effect of substrate elasticity on intracellular trafficking of CD63⁺ MVBs was evaluated. To visualize CD63⁺ MVBs within cells, the red fluorescent protein Katushka2S (K2S) was fused to CD63 and the resulting CD63-K2S fusion was transduced into D1 mouse MSCs. CD63-K2S⁺ MSCs on soft or stiff PEG-DA hydrogels with 0.8 mM RGD were imaged using total internal reflection fluorescence (TIRF) microscopy in order to quantify intracellular transport. Tracked MVB size was not significantly different across tested conditions. CD63-K2S⁺ MVBs were tracked by calculating their ensemble-averaged mean squared displacement (MSD) over time (t). Data were collected every Δt=0.1 sec and fit to the transport equation:

<MSD>=K_{α}t^{α}, (6)

with K_{α} as the transport coefficient and a as the transport exponent. The transport exponent α is ˜1 for Brownian motion when particle transport is unimpeded, and <1 for sub-diffusive, or impeded, transport. The two-dimensional effective diffusion coefficient D_{τ} of tracks was also calculated:

D_{τ}=MSD(τ)/4τ. (7)

Track D_{τ} were calculated over each time interval τ=4Δt=0.4 sec as:

D_{0.4s}=MSD(τ=0.4S)/4(0.4S). (8)

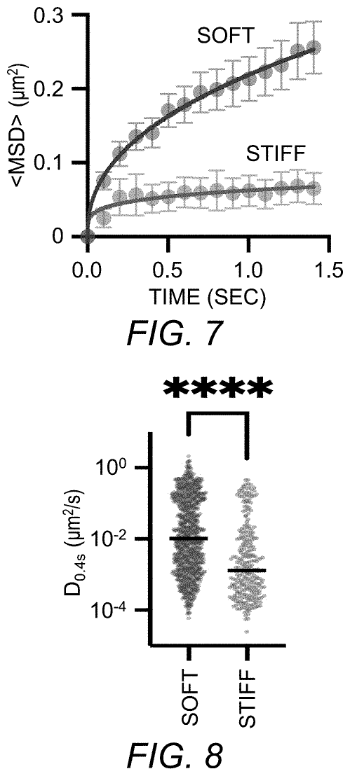

CD63-K2S⁺ MVBs in MSCs transported more rapidly on the soft hydrogel than the stiff hydrogel as indicated by <MSD> vs t plots (FIG. 7) and diffusion coefficient D_{0.4s} (FIG. 8). The transport exponent α across all the conditions was less than 1, indicating that MVB transport is impeded by cytoplasmic contents; exponent α showed a decrease on the stiff hydrogel. Sorting tracks into ‘slow’ or ‘fast’ subgroups revealed that more tracks were considered ‘slow’ for cells on the stiff hydrogel. Thus, softer substrates facilitate intracellular MVB transport.

The impact of cell-matrix interactions on the fusion of MVBs with the plasma membrane was subsequently evaluated. To accomplish this, the pH-sensitive reporter pHLuorin2 was fused to CD63. This reporter will turn on GFP fluorescence when pH changes from low (acidic) to high (neutral) as occurs when acidic CD63⁺ MVBs carrying exosomes fuse with the plasma membrane and release exosomes into the pH-neutral extracellular space. After transducing CD63-pHLuorin2 into D1 mouse MSCs, MSCs seeded on soft or stiff PEG-DA hydrogels with 0.8 mM RGD were imaged over time using the same TIRF microscopy method every Δt=0.1 sec with total time T=25 sec. Flashing events were determined using a custom program (see Methods). The intensity for counted events exhibited a sharp increase followed by a rapid decrease; substrate elasticity does not impact the kinetics of flashing events. MSCs on the stiff hydrogel produced significantly less events than MSCs on the soft hydrogel. Thus, softer hydrogels promote nanoscale biological events that result in increased EV secretion.

### Example 4: Arp2/3 Limits Intracellular MVB Trafficking in MSCs on Substrates

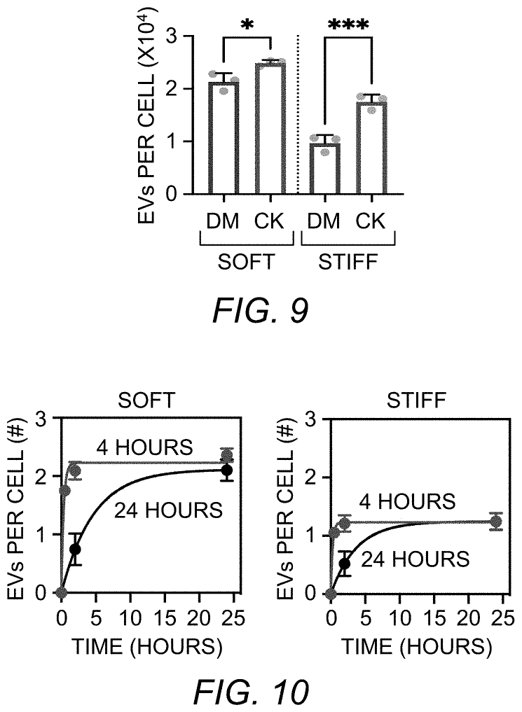

Mechanisms behind how biophysical regulation of cell-matrix interactions impacts EV secretion were assessed. Because myosin-II contractility is known to mediate mechanosensing, it was determined whether its inhibition would rescue EV secretion from MSCs on stiff substrates. Surprisingly, 50 μM blebbistatin, an inhibitor of myosin-II ATPase, did not impact exosome secretion from human MSCs on either soft or stiff elastic alginate-RGD hydrogels. Thus, substrate stiffness-mediated changes in EV secretion do not require myosin-II activity. The average mesh size of intracellular cytoskeleton networks in mesenchymal cell types is typically ˜50 nm for cells cultured on plastic, and hence the mesh likely impedes the transport of MVBs that contain multiple ILVs, which are released as exosomes. Since cells on softer substrates show more fluid-like, less dense actin cytoskeletons, which can be regulated independently of myosin-II, the role of actin networks in EV secretion from MSCs on substrates was investigated. FAK is known to promote actin assembly by interacting with the actin related protein 2/3 (Arp2/3) complex, and inhibition of FAK rescues EV secretion on the stiff hydrogel (FIG. 2). Thus, Arp2/3 in MSCs was inhibited as the cells were just beginning to undergo cell spreading and form actin networks. It was first confirmed that MSCs seeded on substrates for a shorter period of time (˜4 hour) followed by washing and EV collection after 24 hours showed a similar difference in EV number per cell between soft and stiff hydrogels. Treatment of human MSCs with 5 μM CK869, an Arp2/3 inhibitor, increased EV secretion on the stiff hydrogel, and the effect was also observed on the soft hydrogel to a lesser but significant extent (FIG. 9). Arp2/3 inhibition did not change the size of tracked CD63-K2S⁺ MVBs in MSCs on substrates. However, Arp2/3 inhibition significantly enhanced transport on either substrate as indicated by representative tracks, <MSD> vs t plots, and D_{0.4s} while transport exponent α remained unchanged at ˜0.5. Thus, Arp2/3 limits MVB transport and EV secretion on hydrogels.

Changes in actin cytoskeleton structural organization associated with substrate elasticity-directed EV secretion was subsequently characterized. Confocal imaging analysis showed that mean intensity of ALEXA FLUOR® 488 phalloidin-labelled F-actin signal per cell was increased in MSCs on the stiff hydrogel after cell adhesion for 4 hours, while Arp2/3 inhibition by CK869 for 2 hours was sufficient to decrease the signal intensity on both substrates. To further understand changes in nanoscale F-actin organization, super-resolution direct Stochastic Optical Reconstruction Microscopy (dSTORM) imaging was performed using TIRF microscopy, revealing molecular localizations of ALEXA FLUOR® 647 phalloidin-labelled F-actin signal at the interface between the cell and substrate (with super-resolution precision (˜40 nm average localization accuracy) (see Methods). The density-based spatial clustering of applications with noise (DBSCAN) algorithm was then used to determine whether F-actin clusters were present as shorter fragments (<15 localizations per cluster), which were previously observed in red blood cells, or more substantial fibers (>1000 localizations per cluster). Both shorter fragments and fiber F-actin signals were observed when MSCs were seeded on the soft hydrogel, while fibers were more prominent on the stiff hydrogel. CK869 treatment for 2 hours did not alter the distribution of different F-actin clusters on both substrates, suggesting that Arp2/3 mediates the amount of actin polymers per cell, but not actin clustering or bundling within this timescale.

A biophysical factor that can also potentially limit MVB fusion and exosome release is the membrane-cytoskeleton attachment (MCA), which is known to regulate both exocytosis and endocytosis. The MCA can be quantified by the measurement of force for membrane tether formation using atomic force microscopy (AFM) (see Methods). The results showed that substrate elasticity does not impact the force of membrane tether formation within 4 hours of cell adhesion, although CK869 treatment for 2 hours slightly decreased tethering on the soft hydrogel. Together, the presence of larger actin fibers near the cell surface likely impedes MVB transport and fusion within cells on hydrogels.

### Example 5: EV Production Kinetics Depends on Cell Adhesion Time on Substrates

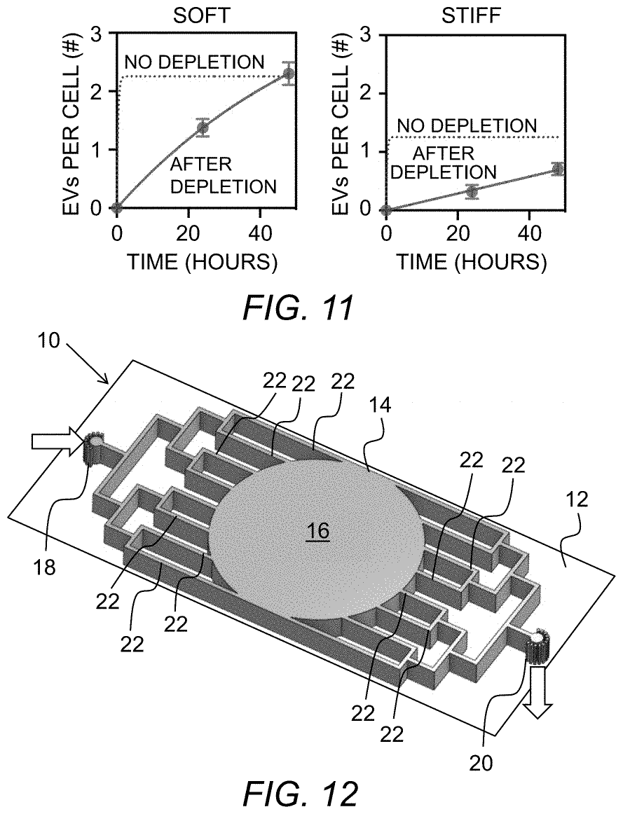

MSCs were seeded on soft (E ˜3 kPa) and stiff (E ˜20 kPa) hydrogels. The MSCs were incubated for either 4 hours or 24 hours before being washed to remove unattached cells. EV production was subsequently assessed for 24 hours. The results of this analysis (FIG. 10) indicated that MSCs on soft substrates reached maximum EV production after only 5 minutes.

To further evaluate shows EV production kinetics, MSCs were seeded on soft (E ˜3 kPa) and stiff (E ˜20 kPa) hydrogels. The MSCs were incubated for 4 hours, washed to remove unattached cells, and EVs produced after 5 minutes were removed from the culture. This analysis indicated that EV-depleted cultures continued to produce EVs for at least 48 hours after depletion (FIG. 11).

Based upon this analysis, EV-producing cells can be readily seeded onto a microfluid device coated with a soft hydrogel substrate (FIG. 12) and EVs can be collected for one or more days from the cells under flow.

### Example 6: MSC EVs from Different Substrates Retain Therapeutic Activity and Cargo Contents Against Acute Lung Injury In Vivo

The results herein collectively show that the combination of a soft (E ˜3 kPa) hydrogel substrate (FIG. 1) and a lower (0.16 mM) RGD concentration (FIG. 5) results in a total ˜10-fold increase in EV secretion per cell than plastic culture. Thus, the potential impact of substrate elasticity on EV content and functionality was evaluated. MSC-derived EVs are known to attenuate acute lung injury. Thus, a matched dose of EVs (6×10⁷ per 20 g mouse) from primary mouse MSCs cultured on soft or stiff PEG-DA hydrogels with 0.16 mM RGD or plastic culture were delivered intratracheally (i.t.) 4 hours after inducing lung injury in mice using lipopolysaccharides (LPS). After 24 hours, a significant reduction in lung edema and vascular permeability as shown by albumin accumulation in lung parenchyma was observed for mice treated with EVs from all tested substrates. At this dose, EVs from MSCs on the soft hydrogel facilitate the reduction of edema and vascular permeability in LPS-treated mice to a greater extent than EVs from MSCs on plastic.

The therapeutic mechanism by which MSC-derived EVs attenuate acute lung injury is reported to require presentation of the CD44 receptor on the EV surface, which is known to facilitate uptake into host monocytes, monocyte-derived macrophages, and alveolar type II cells. Thus, it was determined whether EVs derived from cells on different substrates provide therapeutic efficacy through a similar mechanism that requires uptake by host cells through presence of free CD44 receptor on the EV surface. To accomplish this, the maximum effective dose of EVs (3×10⁸ per 20 g mouse) secreted from MSCs on different substrates were treated with a CD44 blocking antibody, all of which negated their therapeutic activity in terms of lung edema and permeability, demonstrating that MSC-derived EVs from different substrates leverage CD44 to achieve therapeutic efficacy in acute lung injury. EV cargo composition from primary mouse MSCs was also profiled in terms of different molecules known to be essential in ameliorating acute lung injury (e.g., mRNAs such as KGF and IL-6; mtDNAs such as ND1 and ATP6; and miRNA such as miR-146A, miR-30b-3p and miR27a-3p). EVs from different substrates showed a similar level of keratinocyte growth factor (KGF) and interleukin-6 (IL-6) RNAs, mitochondrial DNAs, and miRNAs. Thus, soft hydrogels enhance EV secretion without compromising functionality or cargo contents to resolve injury.

What is claimed is:

1. A method of increasing the production of extracellular vesicles comprising
i) adhering extracellular vesicle-producing cells to a cell-adhesive substrate comprising a hydrogel having a Young's modulus of about 0.1 kPa to about 5 kPa, wherein the hydrogel is functionalized with one or more cell-adhesive peptides;
ii) incubating the extracellular vesicle-producing cells, optionally in the presence of one or more inhibitors of cell migration, actin polymerization, cell-cell adhesion, or cell-substrate adhesion; and
iii) collecting the extracellular vesicles secreted by the extracellular vesicle-producing cells.

2. The method of claim 1, wherein the extracellular vesicle-producing cells comprise mesenchymal stromal cells.

3. The method of claim 1, wherein the extracellular vesicle-producing cells are adhered to the cell-adhesive substrate at a density of at least about 25 to about 150 cells per mm².

4. The method of claim 1, wherein the hydrogel comprises alginate, chitosan, polyethylene glycol, polyacrylamide, gelatin, collagen, laminin, elastin, hyaluronic acid, decellularized scaffold from tissue, or a solubilized basement membrane preparation extracted from the Engelbreth-Holm-Swarm mouse sarcoma.

5. The method of claim 1, wherein the one or more cell adhesive peptides comprise fibronectin peptides, hyaluronic acid peptides, laminin peptides, peptides of a matricellular protein, linear RGD peptides, cyclic RGD peptides, PHSRN (SEQ ID NO:28) peptides, CD44-binding peptides, α6β1 integrin binding peptides, α3β1 integrin binding peptides, α6β4 integrin binding peptides, heparin-binding domain-derived peptides from thrombospondin, tenascin-C-derived peptides, or SPARC-derived peptides.

6. The method of claim 1, wherein the one or more inhibitors of cell migration, actin polymerization, cell-cell adhesion, or cell-substrate adhesion is an inhibitor of focal adhesion kinase, inhibitor of actin related protein 2/3, inhibitor of actin, inhibitor of a cadherin, or inhibitor of an integrin.

7. The method of claim 1, wherein step iii) is repeated 2 or more times in a 24 hour period.

8. The method of claim 1, wherein step iii) is carried out continuously.

9. A microfluidic device comprising one or more flow channels for collecting extracellular vesicles from extracellular vesicle-producing cells and a compartment comprising a cell-adhesive substrate comprising a hydrogel having a Young's modulus of about 0.1 kPa to about 5 kPa, wherein the hydrogel is functionalized with one or more cell adhesive peptides.

10. The microfluidic device of claim 9, wherein the substrate has a thickness of about 5 μm to about 5 mm.

11. The microfluidic device of claim 9, wherein the hydrogel comprises alginate, chitosan, polyethylene glycol, polyacrylamide, gelatin, collagen, laminin, elastin, hyaluronic acid, decellularized scaffold from tissue, or a solubilized basement membrane preparation extracted from the Engelbreth-Holm-Swarm mouse sarcoma.

12. The microfluidic device of claim 9, wherein the one or more cell adhesive peptides comprise fibronectin peptides, hyaluronic acid peptides, laminin peptides, peptides of a matricellular protein, linear RGD peptides, cyclic RGD peptides, PHSRN (SEQ ID NO:28) peptides, CD44-binding peptides, α6β4 integrin binding peptides, α3β1 integrin binding peptides, α6β4 integrin binding peptides, heparin-binding domain-derived peptides from thrombospondin, tenascin-C-derived peptides, or SPARC-derived peptides.
